(19) United States
(12) Patent Application Publication
(10) Pub. No.: US 2025/0025110 A1
(43) Pub. Date: Jan. 23, 2025

(54) INFORMATION PROCESSING APPARATUS, INFORMATION PROCESSING METHOD, AND INFORMATION PROCESSING PROGRAM

(71) Applicant: FUJIFILM CORPORATION, Tokyo (JP)

(72) Inventor: Yuki WADA, Tokyo (JP)

(21) Appl. No.: 18/907,591

(22) Filed: Oct. 7, 2024

Related U.S. Application Data
(63) Continuation of application No. PCT/JP2023/013729, filed on Mar. 31, 2023.

(30) Foreign Application Priority Data
Apr. 18, 2022 (JP) 2022-068440

(51) Int. Cl. A61B 5/00; G16H 10/60; G16H 15/00; G16H 20/00

(52) U.S. Cl. CPC A61B 5/742; G16H 10/60; G16H 15/00; G16H 20/00

(57) ABSTRACT

An information processing apparatus includes at least one processor. The processor is configured to acquire a first medical document in which information on a disease recognized for a patient is described, and acquire a second medical document in which information on a treatment for the disease of the patient or information on course information on the disease of the patient is described. The processor is configured to display, for each target disease including the disease described in the first medical document and the disease described in the second medical document, first information indicating that description about the target disease is present in the first medical document and second information indicating that description about the target disease is present in the second medical document in an identifiable manner.

### CROSS-REFERENCE TO RELATED APPLICATIONS

This application is a continuation application of International Application No. PCT/JP2023/013729, filed Mar. 31, 2023, the disclosure of which is incorporated herein by reference in its entirety. Further, this application claims priority from Japanese Patent Application No. 2022-068440, filed on Apr. 18, 2022, the disclosure of which is incorporated herein by reference in its entirety.

### BACKGROUND

### 1. Technical Field

The technology of the present disclosure relates to an information processing apparatus, an information processing method, and an information processing program.

### 2. Description of the Related Art

As a technology for preventing overlooking of matters described in an interpretation report, the following technology is known. For example, JP2020-38429A discloses a medical support device comprising a medical report acquisition unit that acquires a medical report, an importance degree determination unit that determines an importance degree of the medical report, a distribution destination determination unit that determines a distribution destination of an alert related to the medical report by using the importance degree, and a distribution unit that distributes the alert to the distribution destination determined by the distribution destination determination unit.

JP2019-204189A discloses an interpretation report management device comprising a creation unit that extracts a predetermined character string from a first interpretation report created by an interpretation doctor in response to an interpretation request from a clinical doctor and creates a second interpretation report in which a checkbox is provided at a position corresponding to the extracted predetermined character string in the first interpretation report, a check determination unit that determines whether or not a check is put in all the checkboxes provided in the second interpretation report, and a reception unit that receives a display end request of the second interpretation report in a case in which the check is put in all the checkboxes provided in the second interpretation report.

### SUMMARY

Information on a disease extracted from a medical image, such as a plain X-ray image, a computed tomography (CT) image, a mammography image, or a magnetic resonance imaging (MRI) image, is described in an interpretation report. A doctor performs appropriate treatment for the disease described in the interpretation report. In a case in which the disease described in the interpretation report is overlooked, a serious incident, such as a delay in treatment for the disease and a worsening of symptoms, may occur.

The technology of the present disclosure has been made in view of the above-described point, and an object thereof is to prevent the overlooking of a disease described in a medical document such as an interpretation report.

The technology of the present disclosure relates to an information processing apparatus including at least one processor. The processor is configured to acquire a first medical document in which information on a disease recognized for a patient is described, and acquire a second medical document in which information on a treatment for the disease of the patient or information on course information on the disease of the patient is described. The processor is configured to display, for each target disease including the disease described in the first medical document and the disease described in the second medical document, first information indicating that description about the target disease is present in the first medical document and second information indicating that description about the target disease is present in the second medical document in an identifiable manner.

According to the technology of the present disclosure, it is possible to prevent the overlooking of the disease described in the medical document such as the interpretation report.

### BRIEF DESCRIPTION OF THE DRAWINGS

Exemplary embodiments according to the technique of the present disclosure will be described in detail based on the following figures, wherein:

FIG. 1 is a diagram showing an example of a configuration of a medical information management system according to an embodiment of the technology of the present disclosure;

FIG. 2 is a diagram showing an example of a hardware configuration of an information processing server according to the embodiment of the technology of the present disclosure;

FIG. 3 is a functional block diagram showing an example of a functional configuration of the information processing server according to the embodiment of the technology of the present disclosure;

FIG. 4 is a diagram showing an example of a chart according to the embodiment of the technology of the present disclosure;

FIG. 5 is a diagram showing an example of the chart according to the embodiment of the technology of the present disclosure;

FIG. 6 is a diagram showing an example of the chart according to the embodiment of the technology of the present disclosure; and

FIG. 7 is a flowchart showing an example of a flow of processing performed by executing an information processing program via a CPU according to the embodiment of the technology of the present disclosure.

### DETAILED DESCRIPTION

Hereinafter, an example of an embodiment of the technology of the present disclosure will be described with reference to the accompanying drawings. It should be noted that the same or equivalent components and parts in the respective drawings are denoted by the same reference numerals, and the duplicated description will be omitted.

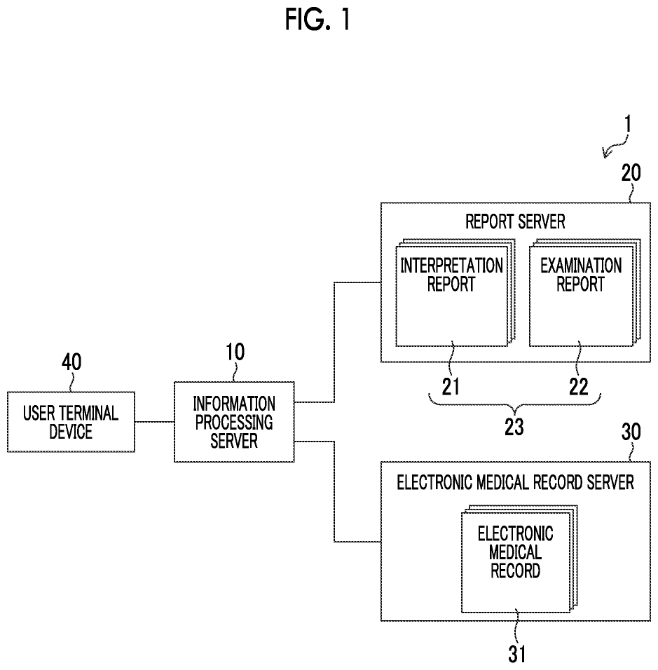

FIG. 1 is a diagram showing an example of a configuration of a medical information management system 1 according to the embodiment of the technology of the present disclosure. The medical information management system 1 has a configuration including an information processing server 10, a report server 20, an electronic medical record server 30, and a user terminal device 40.

The report server 20 is a server computer comprising a storage medium that stores medical documents such as an interpretation report 21 and an examination report 22. In the interpretation report 21, for example, information on a disease extracted from medical images, such as a plain X-ray image, a CT image, a mammography image, or an MRI image, is described. In the examination report 22, for example, information on a result of an examination performed on a tissue or an organ collected from a patient is described. The interpretation report 21 and the examination report 22 are created for each patient. A plurality of interpretation reports 21 and a plurality of examination reports 22 may be created for one patient. It should be noted that, in a case in which the distinction between the interpretation report 21 and the examination report 22 is not made or the distinction between the interpretation report 21 are collectively referred to, the interpretation report 21 and the examination report 22 are simply referred to as “report 23” in the following description. The report 23 is an example of a “first medical document” according to the technology of the present disclosure.

The electronic medical record server 30 is a server computer comprising a storage medium that stores the electronic medical record 31 created for each patient. The electronic medical record 31 includes information on a treatment for a disease of the patient or course information on the disease of the patient. Examples of the “information on the treatment” include subjective information, objective information, assessment, plan, a medication status, an examination result, an examination order, and the like for the disease.

A doctor who creates the electronic medical record 31 registers the disease described in the report 23 as a disease that needs to be treated for the patient by using a registration system (not shown). The registered disease is recorded in the electronic medical record 31 through system linkage. The disease includes a positive disease that is determined in a definitive manner and a suspected disease (false positive disease) that is not determined in a definitive manner but is suspected as being positive. Information indicating whether the disease is the positive disease or the suspected disease is assigned to each disease described in the electronic medical record 31. The electronic medical record 31 is an example of a “second medical document” according to the technology of the present disclosure.

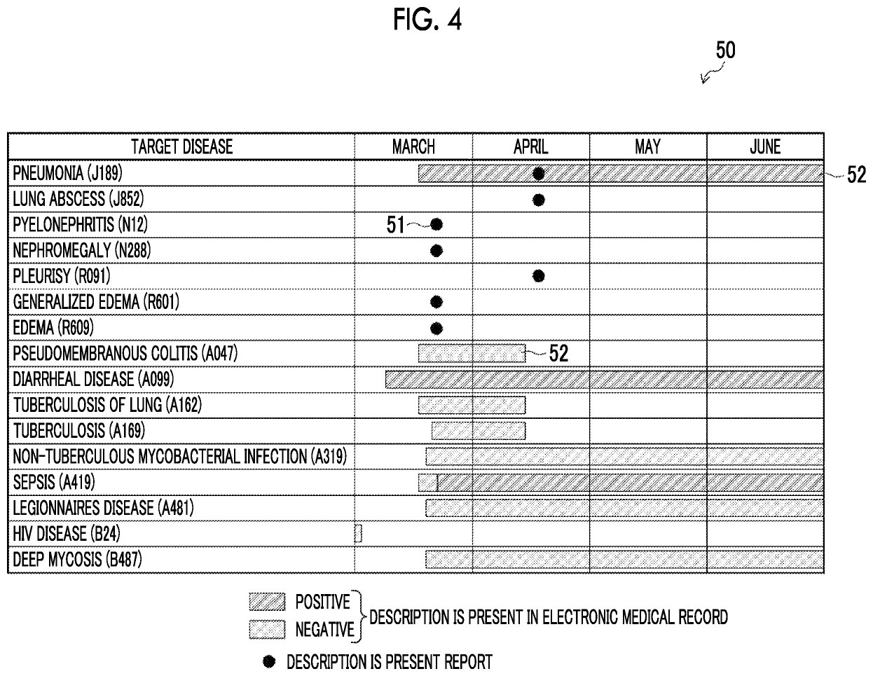

The information processing server 10 aggregates the diseases described in the report 23 and the diseases described in the electronic medical record 31 for each patient, and generates a chart 50 (see FIG. 4) to enable an overarching perspective confirmation of a description status in the report 23 and the electronic medical record 31 for these diseases. The information processing server 10 is an example of an “information processing apparatus” according to the technology of the present disclosure.

The user terminal device 40 is a computer intended to be used by a doctor. The user terminal device 40 issues a generation request of the chart 50 for a specific patient to the information processing server 10. The information processing server 10 generates the chart 50 for the specific patient based on the request from the user terminal device 40 and transmits the generated chart 50 to the user terminal device 40. The chart 50, which is transmitted from the information processing server 10, is displayed on a display (not shown) of the user terminal device 40.

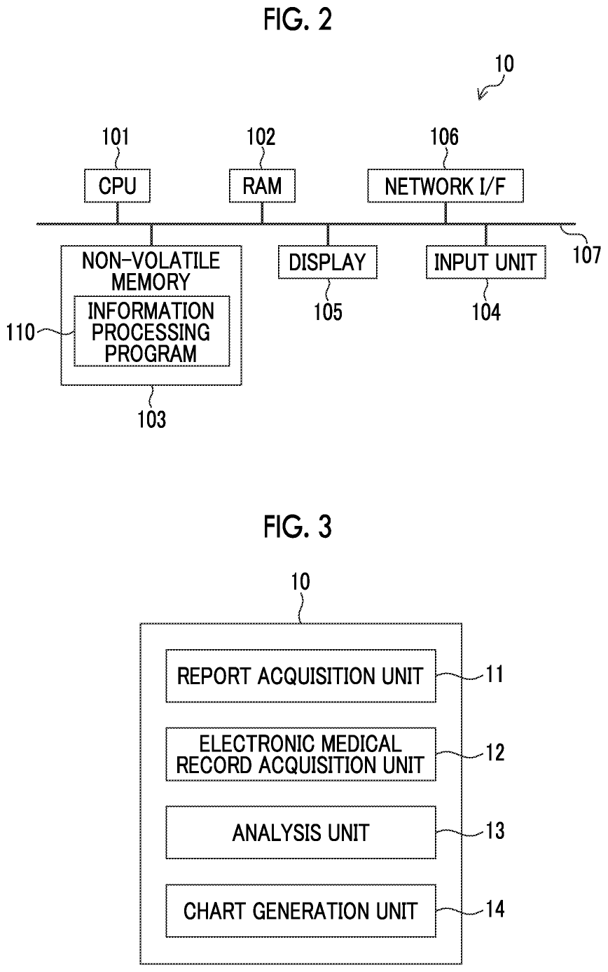

FIG. 2 is a diagram showing an example of a hardware configuration of the information processing server 10. The information processing server 10 includes a central processing unit (CPU) 101, a random access memory (RAM) 102, a non-volatile memory 103, an input device 104, a display 105, and a network interface 106. These pieces of hardware are connected to a bus 107.

The display 105 is, for example, a liquid crystal display or a light emitting diode (LED) display. The input device 104 includes, for example, a keyboard and a mouse, and may include a proximity input device such as a touch panel display, a voice input device such as a microphone, and a contactless input device such as a camera or a sensor. The network interface 106 is an interface for connecting the information processing server 10 to the report server 20, the electronic medical record server 30, and the user terminal device 40 via a network.

The non-volatile memory 103 is a non-volatile storage medium such as a hard disk and a flash memory. The non-volatile memory 103 stores an information processing program 110. The RAM 102 is a work memory for the CPU 101 to execute processing. The CPU 101 loads the information processing program 110 stored in the non-volatile memory 103 into the RAM 102, and executes the processing according to the information processing program 110. The CPU 101 is an example of a “processor” according to the technology of the present disclosure.

FIG. 3 is a functional block diagram showing an example of a functional configuration of the information processing server 10. The information processing server 10 includes a report acquisition unit 11, an electronic medical record acquisition unit 12, an analysis unit 13, and a chart generation unit 14. The CPU 101 executes the information processing program 110, whereby the CPU 101 functions as the report acquisition unit 11, the electronic medical record acquisition unit 12, the analysis unit 13, and the chart generation unit 14.

The report acquisition unit 11 accesses the report server 20 to acquire the report 23 related to the specific patient from among the reports 23 stored in the storage medium provided in the report server 20. The specific patient is designated by, for example, the user terminal device 40. Patient identification information, such as a patient identification (ID), is assigned to the interpretation report 21 and the examination report 22. The report acquisition unit 11 transmits a report acquisition request including the patient identification information (patient ID) to the report server 20. The report server 20 extracts the report to which the patient identification information (patient ID) related to the acquisition request is assigned, from among the reports 23 stored in the storage medium provided in the report server 20, and transmits the extracted report to the information processing server 10. The report acquisition unit 11 acquires the report 23 transmitted from the report server 20. It should be noted that, in a case in which a plurality of reports 23 are created for one patient, all of the reports 23 for the patient are acquired.

The report server 20 may comprise a report database (not shown) in which the patient identification information (patient ID) and identification information (report ID) of the interpretation report 21 and the examination report 22 are associated with each other. In such a case, the report server 20 may extract the report 23 related to the specific patient by using the report database.

The electronic medical record acquisition unit 12 accesses the electronic medical record server 30 to acquire the electronic medical record 31 related to the specific patient from among the electronic medical records 31 stored in the storage medium provided in the electronic medical record server 30. The patient identification information (patient ID) is assigned to the electronic medical record 31. The electronic medical record acquisition unit 12 transmits an electronic medical record acquisition request including the patient identification information (patient ID) to the electronic medical record server 30. The electronic medical record server 30 extracts the electronic medical record 31 to which the patient identification information (patient ID) related to the acquisition request is assigned, from among the electronic medical records 31 stored in the storage medium provided in the electronic medical record server 30, and transmits the extracted electronic medical record 31 to the information processing server 10. The electronic medical record acquisition unit 12 acquires the electronic medical record 31 transmitted from the electronic medical record server 30. It should be noted that, in a case in which a plurality of electronic medical records 31 are created for one patient, all of the electronic medical records 31 for the patient are acquired.

The electronic medical record server 30 may comprise an electronic medical record database (not shown) in which the patient identification information (patient ID) and the identification information (electronic medical record ID) of the electronic medical record 31 are associated with each other. In such a case, the electronic medical record server 30 may extract the electronic medical record 31 related to the specific patient by using the electronic medical record database.

The analysis unit 13 analyzes the text described in the report 23 acquired by the report acquisition unit 11, extracts a description part including a name or a type of the disease described in the report 23, and specifies the disease described in the report 23 based on the extracted description part.

Similarly, the analysis unit 13 analyzes the text described in the electronic medical record 31 acquired by the electronic medical record acquisition unit 12, extracts a description part including a name or a type of the disease described in the electronic medical record 31, and specifies the disease described in the electronic medical record 31 based on the extracted description part. The analysis unit 13 further specifies whether the disease described in the electronic medical record 31 is the positive disease or the suspected disease. Information indicating whether the disease is the positive disease or the suspected disease is assigned to each disease described in the electronic medical record 31. The analysis unit 13 specifies whether the disease specified in the electronic medical record 31 is the positive disease or the suspected disease by referring to the information. It should be noted that, in a case in which the electronic medical record 31 stores, for each electronic medical record 31, the disease in a form for specifying the disease, including the name or the type of the disease, the text analysis or the like is not required.

The chart generation unit 14 generates the chart 50 to enable an overarching perspective confirmation of the description status of the disease specified by the analysis unit 13 in the report 23 and/or the electronic medical record 31. FIG. 4 is a diagram showing an example of the chart 50 generated by the chart generation unit 14.

The chart generation unit 14 generates the chart 50 in which target diseases, which include the disease described in the report 23 and the disease described in the electronic medical record 31 and are specified by the analysis unit 13, are taken on a first axis (vertical axis), and dates are taken on a second axis (horizontal axis) intersecting the first axis. The target disease may be classified according to the International Classification of Diseases, 10th Revision (ICD-10).

The chart generation unit 14 displays, for each target disease, first information 51 indicating that the description about the target disease is present in the report 23 and second information 52 indicating that the description about the target disease is present in the electronic medical record 31 on the chart 50 in an identifiable manner. In the example shown in FIG. 4, the first information 51 is shown by a circular marker, and the second information 52 is shown by a bar graph. The chart generation unit 14 displays the first information 51 on the chart 50 along with information indicating a point in time when the target disease is described in the report 23, and displays the second information 52 along with information indicating a point in time when the target disease is described in the electronic medical record 31.

That is, the type or the name of the disease described in the report 23 is indicated by the position of the marker as the first information 51 on the first axis, and the point in time when the disease is described in the report 23 is indicated by the position of the marker as the first information 51 on the second axis. According to the chart 50 shown in FIG. 4 as an example, for a certain patient, the information on pyelonephritis (N12), nephromegaly (N288), generalized edema (R601), and edema (R609) is described in the report 23 in March, and the information on pneumonia (J189), lung abscess (J852), and pleurisy (R091) is described in the report 23 in April. The chart generation unit 14 acquires information indicating the point in time when each disease specified by the analysis unit 13 is described in the report 23, and determines the position of the marker based on this information. The information indicating the point in time when the disease is described in the report 23 may be date information assigned to report 23.

A type or a name of the disease described in the electronic medical record 31 is indicated by the position of the bar graph as the second information 52 on the first axis. A start point of the bar graph indicates the point in time when the disease is described in the electronic medical record 31 (in other words, a point in time when the disease is registered as a treatment target). An end point of the bar graph indicates a point in time when the disease is deleted from the electronic medical record 31 (in other words, a point in time when the registration of the disease as the treatment target is released). The electronic medical record 31 is assumed to be updated daily. The bar graph indicates that the description about the disease is continuously present in the electronic medical record 31. According to the chart 50 shown in FIG. 4 as an example, the information on pneumonia (J189) is first described in the electronic medical record 31 in March, and this description is continuously present in the electronic medical record 31 up to the present. The chart generation unit 14 acquires the information indicating the point in time when each disease specified by the analysis unit 13 is described in the electronic medical record 31 and the point in time when each disease is deleted, and determines the start point and the end point of the bar graph based on this information. The information indicating the point in time when the disease is described in the electronic medical record 31 and the point in time when the disease is deleted may be date information assigned to the electronic medical record 31.

The chart generation unit 14 displays the information indicating that the disease is the positive disease and the information indicating that the disease is the suspected disease, which are included in the second information 52, on the chart 50 in an identifiable manner. For example, the information indicating that the disease is the positive disease and the information indicating that the disease is the suspected disease, which are included in the second information 52, may be displayed using bar graphs of different colors. According to the chart 50 shown in FIG. 4 as an example, in the electronic medical record 31, pneumonia (J189) is described as the positive disease and pseudomembranous colitis (A047) is described as the suspected disease. In addition, sepsis (A419) is initially described as the suspected disease and is later described as the positive disease. The chart generation unit 14 identifies and displays whether the second information 52 is positive or suspected, based on the result of specifying, via the analysis unit 13, whether the disease is the positive disease or the suspected disease for each disease described in the electronic medical record 31.

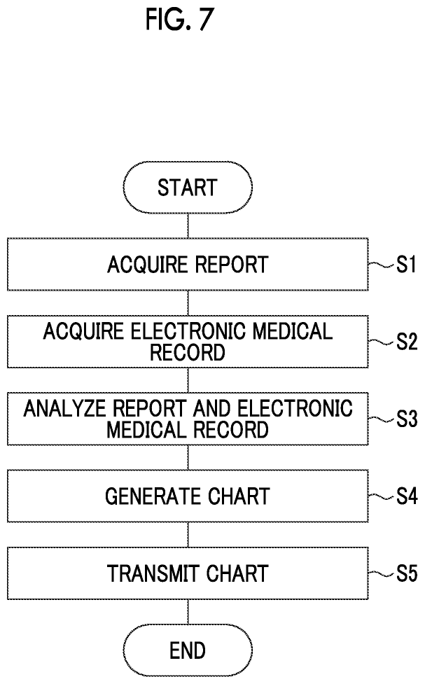

FIG. 7 is a flowchart showing an example of a flow of processing performed by executing the information processing program 110 via the CPU 101 according to the embodiment of the technology of the present disclosure. The information processing program 110 is executed, for example, in a case in which the generation request of the chart 50 for the specific patient is issued from the user terminal device 40.

In step S1, the report acquisition unit 11 accesses the report server 20 to acquire the report 23 related to the specific patient from among the reports 23 stored in the storage medium provided in the report server 20. Specifically, the report acquisition unit 11 transmits the report acquisition request including the patient identification information (patient ID) to the report server 20. The report server 20 extracts the report to which the patient identification information (patient ID) related to the acquisition request is assigned, from among the reports 23 stored in the storage medium provided in the report server 20, and transmits the extracted report to the information processing server 10. The report acquisition unit 11 acquires the report 23 transmitted from the report server 20.

In step S2, the electronic medical record acquisition unit 12 accesses the electronic medical record server 30 to acquire the electronic medical record 31 related to the specific patient from among the electronic medical records 31 stored in the storage medium provided in the electronic medical record server 30. Specifically, the electronic medical record acquisition unit 12 transmits the electronic medical record acquisition request including the patient identification information (patient ID) to the electronic medical record server 30. The electronic medical record server 30 extracts the electronic medical record to which the patient identification information (patient ID) related to the acquisition request is assigned, from among the electronic medical records 31 stored in the storage medium provided in the electronic medical record server 30, and transmits the extracted electronic medical record 31 to the information processing server 10. The electronic medical record acquisition unit 12 acquires the electronic medical record 31 transmitted from the electronic medical record server 30. It should be noted that a case is described in which the report 23 is acquired before the electronic medical record 31 is acquired, but the present disclosure is not limited to this. The report 23 may be acquired after the electronic medical record 31 is acquired, or the acquisition of the report 23 and the acquisition of the electronic medical record 31 may be performed in parallel.

In step S3, the analysis unit 13 analyzes the text described in the report 23 acquired in step S1, and extracts the description part including the name or the type of the disease described in the report 23. The analysis unit 13 specifies the disease described in the report 23 based on the extracted description part. Similarly, the analysis unit 13 analyzes the text described in the electronic medical record 31 acquired in step S2, and extracts the description part including the name or the type of the disease described in the electronic medical record 31. The analysis unit 13 specifies the disease described in the electronic medical record 31 based on the extracted description part. The analysis unit 13 further specifies whether the disease described in the electronic medical record 31 is the positive disease or the suspected disease.

In step S4, the chart generation unit 14 generates the chart 50 based on the analysis result in step S3. That is, the chart generation unit 14 generates the chart 50 in which the target diseases, which include the disease described in the report 23 and the disease described in the electronic medical record 31 and are specified in step S3, are taken on a first axis (vertical axis), and dates are taken on a second axis (horizontal axis) intersecting the first axis. The chart generation unit 14 displays, for each target disease, the first information 51 indicating that the description about the target disease is present in the report 23 and the second information 52 indicating that the description about the target disease is present in the electronic medical record 31 on the chart 50 in an identifiable manner.

The chart generation unit 14 acquires information indicating the point in time when each disease specified in step S3 is described in the report 23, and determines the position of the marker as the first information 51 based on this information. In addition, the chart generation unit 14 acquires the information indicating the point in time when each disease specified in step S3 is described in the electronic medical record 31 and the point in time when each disease is deleted, and determines the start point and the end point of the bar graph as the second information 52 based on this information. In addition, the chart generation unit 14 identifies and displays whether the second information 52 is positive or suspected, based on the result of specifying whether the disease is the positive disease or the suspected disease for each disease described in the electronic medical record 31.

In step S5, the chart generation unit 14 transmits the chart 50 generated in step S4 to the user terminal device 40. The chart 50, which is transmitted from the information processing server 10, is displayed on the display (not shown) of the user terminal device 40.

As described above, the information processing server 10 according to the embodiment of the technology of the present disclosure acquires the report 23 in which the information on the disease recognized for the patient is described. In addition, the information processing server 10 acquires the electronic medical record 31 in which the information on the treatment for the disease of the patient or the course information on the disease of the patient is described. The information processing server 10 displays, for each target disease, including the disease described in the report 23 and the disease described in the electronic medical record 31, the first information 51 indicating that the description about the target disease is present in the report 23 and the second information 52 indicating that the description about the target disease is present in the electronic medical record 31 in an identifiable manner.

As described above, the doctor who creates the electronic medical record 31 registers the disease described in the report 23 as the disease that needs to be treated for the patient by using the registration system (not shown). The registered disease is recorded in the electronic medical record 31 through system linkage. In a case in which the disease described in the report 23 is overlooked, the disease may not be recorded in the electronic medical record 31 and may remain unrecognized as the treatment target. That is, the disease described in the report 23 is recognized as the treatment target in a case in which the disease is described in the electronic medical record 31, but there is a concern that the disease is not recognized as the treatment target when the disease is not described in the electronic medical record 31.

With the information processing server 10 according to the present embodiment, the first information 51 indicating that the description about the target disease is present in the report 23 and the second information 52 indicating that the description about the target disease is present in the electronic medical record 31 are displayed in an identifiable manner. As a result, in a case in which the description about a certain disease is present in the report 23 but is not present in the electronic medical record 31, that is, in a case in which it is presumed that the disease described in the report 23 is overlooked, it is possible to easily understand that such a situation has occurred. That is, with the information processing server 10 according to the embodiment of the technology of the present disclosure, it is possible to prevent the overlooking of the disease described in the medical document such as the image interpretation report.

In addition, with the information processing server 10, the first information 51 is displayed along with the information indicating the point in time when the target disease is described in the report 23, and the second information 52 is displayed along with the information indicating the point in time when the target disease is described in the electronic medical record 31. As a result, it is possible to understand a mutual relationship between the timings when the target disease is described in the report 23 and the electronic medical record 31.

In addition, the first information 51 and the second information 52 are displayed on the chart 50 in which the target disease is taken on the first axis and the date is taken on the second axis intersecting the first axis. As a result, it is possible to enable an overarching perspective confirmation of the description status of the target disease in the report 23 and the electronic medical record 31, including the progress.

In addition, the information indicating that the disease is the positive disease and the information indicating that the disease is the suspected disease, which are included in the second information 52, are displayed on the chart 50 in an identifiable manner. As a result, it is possible to understand a diagnostic status of the disease described in the electronic medical record 31.

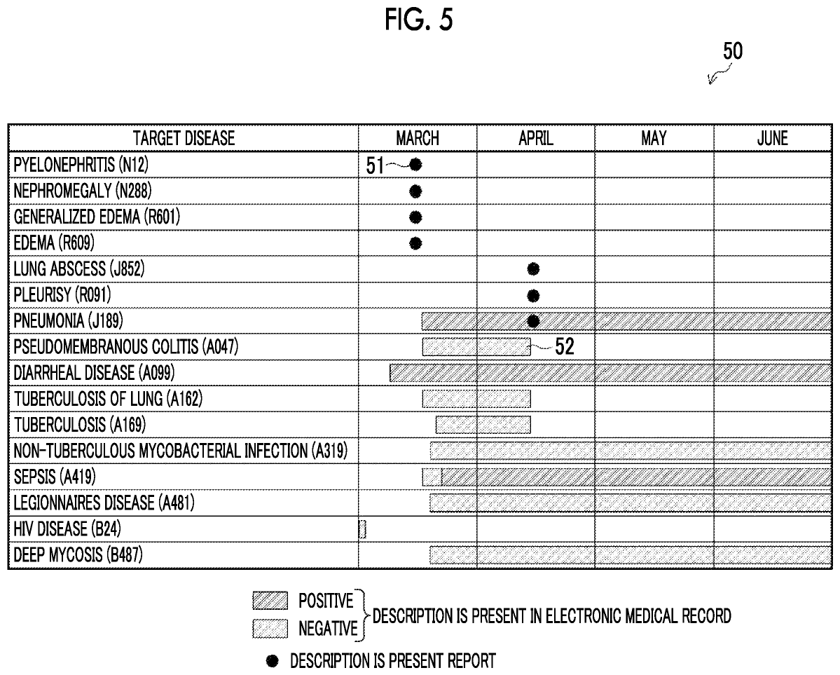

It should be noted that various modifications can be made to the chart 50 shown in FIG. 4. For example, in a case of displaying the target diseases in an arranged manner on the first axis, the target diseases may be displayed in an arranged manner in an order in accordance with the presence or absence of the second information 52 for the target diseases. For example, as shown in FIG. 5, among the target diseases, the target disease that is not described in the electronic medical record 31 may be disposed in the upper part of the chart 50, and the target disease that is described in the electronic medical record 31 may be disposed in the lower part of the chart 50. In addition, among the target diseases, as the target disease that is not described in the electronic medical record 31, the target disease with old date information associated with the report 23 in which the target disease is described may be preferentially displayed. Here, the preferential display indicates that the target disease with old date information is more easily visually recognized by the user than the target disease with new date information. The preferential display may be, for example, display in which the target diseases are arranged in an order from old date information associated with the report 23 in which the target disease is described. For example, as shown in FIG. 5, pyelonephritis and the like described in the report 23 in March may be disposed above lung abscess and the like described in the report 23 in April. In this way, it is possible to preferentially display a disease that is discovered at an earlier stage and is more likely to be overlooked. In addition, a display aspect of the target disease with old date information may be displayed differently from a display aspect of the target disease with new date information. The display aspect of the target disease includes, for example, a color, a pattern, or a shape of a row or a marker of the target disease. For example, the row or the marker of the target disease with old date information may be displayed in red, and the row or the marker of the target disease with new date information may be displayed in black. In particular, by setting the display aspect of the target disease with old date information to be more visually recognizable than the display aspect of the target disease with new date information, it is possible to preferentially display the disease that is discovered at an earlier stage and is more likely to be overlooked.

In addition, in a case in which the target diseases are displayed in an arranged manner along the first axis, the target diseases may be displayed in an arranged manner in an order in accordance with an importance degree of the target disease. For example, among the target diseases, the target disease with relatively high importance degree may be disposed in the upper part of the chart 50, and the target disease with relatively low importance degree may be disposed in the lower part of the chart 50. The importance degree of the target disease may be set in consideration of, for example, at least any one of a risk of severity, resources required for the treatment, a progression degree of the disease, or any combination of a plurality of these factors. In addition, the information processing server 10 may have, for example, an importance degree table in which the importance degree is associated in advance with each disease, derive the importance degree of the disease extracted from the report 23 or the electronic medical record 31 based on the importance degree table, and determine a display order based on these importance degrees. The importance degree table may be set in advance or may be set such that the corresponding importance degree for each disease can be edited in response to the designation or the input of the user.

In addition, in the present embodiment, a case is described in which the second information 52 is displayed in a form of the bar graph, but the second information 52 may be displayed in a form of the marker in the same manner as the first information 51. In such a case, each marker as the second information 52 may indicate an update timing of the electronic medical record 31. Each marker as the second information 52 is displayed in a shape or color different from that of each marker as the first information 51.

In addition, in the present embodiment, a form is described in which the target diseases are displayed in an arranged manner on the first axis on the chart 50, but a target organ, which is an organ related to the target disease, may be displayed in an arranged manner on the first axis on the chart 50 instead of the target disease. In such a case, the analysis unit 13 analyzes the text described in the report 23 acquired by the report acquisition unit 11, extracts the description part including the organ described in the report, and specifies the organ described in the report based on the extracted description part. Similarly, the analysis unit 13 analyzes the text described in the electronic medical record 31 acquired by the electronic medical record acquisition unit 12, extracts a description part including a name of the organ described in the electronic medical record 31, and specifies the organ described in the electronic medical record 31 based on the extracted description part. The chart generation unit 14 generates the chart to enable an overarching perspective confirmation of the description status of the organ specified by the analysis unit 13 in the report 23 and/or the electronic medical record 31. The chart generation unit 14 generates the chart in which target organs, which include the organ described in the report 23 and the organ described in the electronic medical record 31 and are specified by the analysis unit 13, are taken on a first axis (vertical axis), and dates are taken on a second axis (horizontal axis) intersecting the first axis. The chart generation unit 14 displays, for each target organ, first information indicating that the description about the target organ is present in the report 23 and second information 52 indicating that the description about the target organ is present in the electronic medical record 31 on the chart in an identifiable manner.

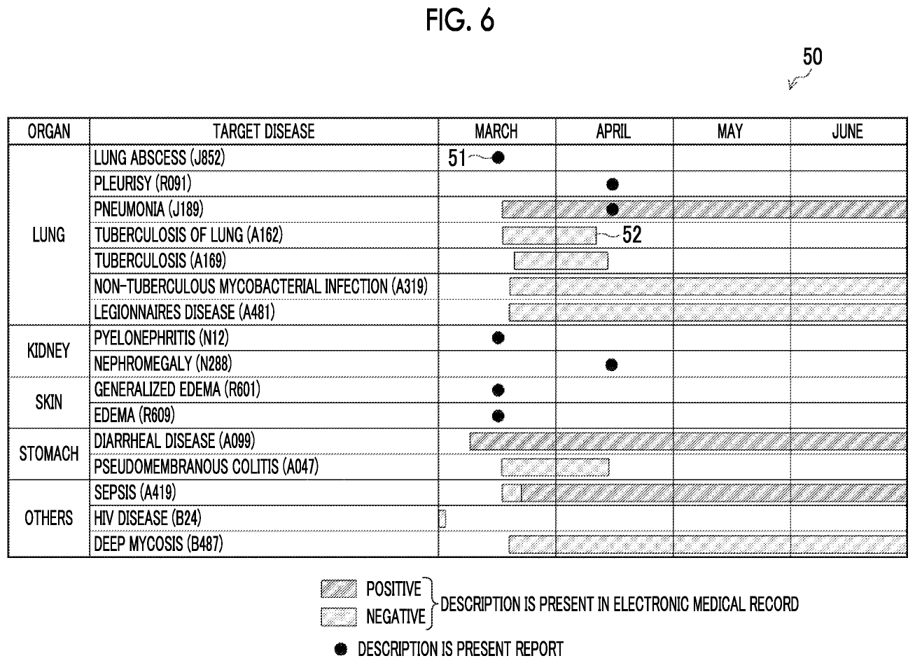

Further, the target diseases may be displayed in an arranged manner on the first axis for each target organ. For example, the information processing server 10 may specify the target diseases and the target organs from the report 23 and the electronic medical record 31, and collectively display the target diseases for each target organ. For example, as shown in FIG. 6, lung abscess, pneumonia, and the like, as the target diseases in which the target organ is the lung, are disposed in a nearby row, and pyelonephritis, nephromegaly, and the like, as the target diseases in which the target organ is the kidney, are disposed in a nearby row. In this way, it is easier to understand the disease associated with each organ. In addition, even in such a case, as in FIG. 5, a determination may be made by distinguishing the organs in a case in which the target diseases are displayed in an arranged manner in an order from old date information associated with the report 23 in which the target disease is described. For example, as shown in FIG. 6, the target diseases in which the target organ is the lung may be displayed in an order from older date information associated with the report 23, and the target diseases in which the target organ is the kidney may be displayed in an order from older date information associated with the report 23.

In the present embodiment, a case is described in which the information processing server 10 generates the chart 50 by executing the information processing program 110, but the user terminal device 40 may generate the chart 50 by executing the information processing program 110. In such a case, the user terminal device 40 functions as an “information processing apparatus” according to the technology of the present disclosure. In such a case, the user terminal device 40 may acquire the report 23 and the electronic medical record 31 used for the generation of the chart 50 via the information processing server 10. In addition, the user terminal device 40 may directly access the report server 20 and the electronic medical record server 30 to acquire the report 23 and the electronic medical record 31 used for the generation of the chart 50. In such a case, since the user terminal device 40 also has a function as the information processing server 10, the information processing server 10 can be omitted in the medical information management system 1. Further, the medical information management system 1 consisting of the information processing server 10 and the user terminal device 40 may correspond to an “information processing apparatus”. For example, the information processing server 10 acquires the report 23 and the electronic medical record 31, specifies the target disease by performing the text analysis on at least the report 23, and transmits the information used for the generation of the chart 50, including the target disease, the date information, and the like in the report 23 and the electronic medical record 31, to the user terminal device 40. The user terminal device 40 may generate the chart 50 based on the information received from the information processing server 10, and display the generated chart 50.

In the above-described embodiment, for example, as the hardware structure of the processing units that execute various types of processing, such as the report acquisition unit 11, the electronic medical record acquisition unit 12, the analysis unit 13, and the chart generation unit 14, various processors shown below can be used. The various processors include, in addition to a CPU and a GPU which are general-purpose processors that execute software (program) and function as various processing units as described above, a programmable logic device (PLD) which is a processor of which a circuit configuration can be changed after manufacture, such as an FPGA, and a dedicated electric circuit which is a processor of which a circuit configuration is designed for exclusive use in order to execute specific processing, such as an application specific integrated circuit (ASIC).

One processing unit may be configured by one of the various processors or may be configured by combining two or more processors of the same type or different types (for example, by combining a plurality of FPGAs or combining a CPU and an FPGA). A plurality of processing units may be configured by one processor.

A first example of the configuration in which the plurality of processing units are configured by one processor is a form in which one processor is configured by combining one or more CPUs and the software and this processor functions as the plurality of processing units, as represented by computers such as a client and a server. A second example thereof is a form in which a processor that realizes the function of the entire system including the plurality of processing units by one integrated circuit (IC) chip is used, as represented by a system on chip (SoC) or the like. In this way, various processing units are configured by one or more of the various processors as the hardware structure.

Further, the hardware structure of the various processors is, more specifically, an electric circuit (circuitry) in which circuit elements such as semiconductor elements are combined.

Moreover, in the above-described embodiment, an aspect is described in which the information processing program 110 is stored (installed) in advance in the non-volatile memory 103, but the technology of the present disclosure is not limited to this. The information processing program 110 may be provided in a form of being recorded in the recording medium such as a compact disc read only memory (CD-ROM), a digital versatile disc read only memory (DVD-ROM), and a universal serial bus (USB) memory. The information processing program 110 may be provided in a form being downloaded from an external device via a network. That is, the program (that is, a program product) described in the present embodiment may be provided using the recording medium or may be distributed from an external computer.

In regard to the above-described embodiment, the following supplementary notes will be further disclosed.

### Supplementary Note 1

An information processing apparatus including: at least one processor, in which the processor is configured to: acquire a first medical document in which information on a disease recognized for a patient is described; acquire a second medical document in which information on a treatment for the disease of the patient or information on course information on the disease of the patient is described; and display, for each target disease including the disease described in the first medical document and the disease described in the second medical document, first information indicating that description about the target disease is present in the first medical document and second information indicating that description about the target disease is present in the second medical document in an identifiable manner.

### Supplementary Note 2

The information processing apparatus according to supplementary note 1, in which the processor is configured to: display the first information along with information indicating a point in time when the target disease is described in the first medical document; and display the second information along with information indicating a point in time when the target disease is described in the second medical document.

### Supplementary Note 3

The information processing apparatus according to supplementary note 2, in which the processor is configured to: generate a chart in which the target disease is taken on a first axis and a date is taken on a second axis intersecting the first axis; and display the first information and the second information on the chart.

### Supplementary Note 4

The information processing apparatus according to any one of supplementary notes 1 to 3, in which the processor is configured to: extract a description part including names or types of the diseases described in the first medical document and the second medical document, and specify the diseases described in the first medical document and the second medical document based on the extracted description part.

### Supplementary Note 5

The information processing apparatus according to any one of supplementary notes 1 to 4, in which the processor is configured to: display information in a case in which the disease is positive and information in a case in which the disease is suspected, in the second information, in an identifiable manner.

### Supplementary Note 6

The information processing apparatus according to supplementary note 3, in which the processor is configured to: display the target diseases in an arranged manner in an order in accordance with the presence or absence of the second information on the target disease.

### Supplementary Note 7

The information processing apparatus according to supplementary note 3, in which the processor is configured to: display the target diseases in an arranged manner in an order in accordance with an importance degree of the target disease.

### Supplementary Note 8

An information processing method including: via at least one processor provided in an information processing apparatus, acquiring a first medical document in which information on a disease recognized for a patient is described; acquiring a second medical document in which information on a treatment for the disease of the patient or information on course information on the disease of the patient is described; and displaying, for each target disease including the disease described in the first medical document and the disease described in the second medical document, first information indicating that description about the target disease is present in the first medical document and second information indicating that description about the target disease is present in the second medical document in an identifiable manner.

### Supplementary Note 9

An information processing program causing at least one processor provided in an information processing apparatus to execute a process including: acquiring a first medical document in which information on a disease recognized for a patient is described; acquiring a second medical document in which information on a treatment for the disease of the patient or information on course information on the disease of the patient is described; and displaying, for each target disease including the disease described in the first medical document and the disease described in the second medical document, first information indicating that description about the target disease is present in the first medical document and second information indicating that description about the target disease is present in the second medical document in an identifiable manner.

It should be noted that the disclosure of JP2022-068440 filed on Apr. 18, 2022 is incorporated in the present specification in its entirety by reference. All of the documents, the patent applications, and the technical standards described in the present specification are incorporated herein by reference to the same extent as in a case in which each document, each patent application, and each technical standard are specifically and individually described by being incorporated in the present specification by reference.

What is claimed is:

1. An information processing apparatus comprising:
at least one processor,
wherein the processor is configured to:
acquire a first medical document in which information on a disease recognized for a patient is described;
acquire a second medical document in which information on a treatment for the disease of the patient or information on course information on the disease of the patient is described; and
display, for each target disease including the disease described in the first medical document and the disease described in the second medical document, first information indicating that description about the target disease is present in the first medical document and second information indicating that description about the target disease is present in the second medical document in an identifiable manner.

2. The information processing apparatus according to claim 1,
wherein the processor is configured to:
display the first information along with information indicating a point in time when the target disease is described in the first medical document; and
display the second information along with information indicating a point in time when the target disease is described in the second medical document.

3. The information processing apparatus according to claim 2,
wherein the processor is configured to:
generate a chart in which the target disease is taken on a first axis and a date is taken on a second axis intersecting the first axis; and
display the first information and the second information on the chart.

4. The information processing apparatus according to claim 1,
wherein the processor is configured to:
extract a description part including names or types of the diseases described in the first medical document and the second medical document, and specify the diseases described in the first medical document and the second medical document based on the extracted description part.

5. The information processing apparatus according to claim 1,
wherein the processor is configured to:
display information in a case in which the disease is positive and information in a case in which the disease is suspected, in the second information, in an identifiable manner.

6. The information processing apparatus according to claim 3,
wherein the processor is configured to:
display the target diseases in an arranged manner in an order in accordance with the presence or absence of the second information on the target disease.

7. The information processing apparatus according to claim 3,
wherein the processor is configured to:
display the target diseases in an arranged manner in an order in accordance with an importance degree of the target disease.

8. An information processing method comprising:
via at least one processor provided in an information processing apparatus,
acquiring a first medical document in which information on a disease recognized for a patient is described;
acquiring a second medical document in which information on a treatment for the disease of the patient or information on course information on the disease of the patient is described; and
displaying, for each target disease including the disease described in the first medical document and the disease described in the second medical document, first information indicating that description about the target disease is present in the first medical document and second information indicating that description about the target disease is present in the second medical document in an identifiable manner.

9. A non-transitory computer-readable storage medium storing an information processing program causing at least one processor provided in an information processing apparatus to execute a process comprising:
acquiring a first medical document in which information on a disease recognized for a patient is described;
acquiring a second medical document in which information on a treatment for the disease of the patient or information on course information on the disease of the patient is described; and
displaying, for each target disease including the disease described in the first medical document and the disease described in the second medical document, first information indicating that description about the target disease is present in the first medical document and second information indicating that description about the target disease is present in the second medical document in an identifiable manner.
